(19) United States
(12) Patent Application Publication
(10) Pub. No.: US 2006/0110366 A1
(43) Pub. Date: May 25, 2006

(54) Entomopathogenic microorganism spores carrier and method for controlling harmful insects

(76) Inventors: Eduardo Primo Yufera, Valencia (ES); Mohamed Ibrahim Fahmy, Valencia (ES); Juan Munoz Pallares, Valencia (ES); Pilar Moya Sanz, Valencia (ES)

Correspondence Address: KLAUBER & JACKSON;J. DAVID SMITH, 411 CONTINENTAL PLAZA, HACKENSACK, NJ, 07601, US

(21) Appl. No.: 10/630,367

(22) Filed: Jul. 30, 2003

Related U.S. Application Data
(63) Continuation of application No. PCT/ES02/00043, filed on Jan. 30, 2002.

(30) Foreign Application Priority Data
Jan. 30, 2001 (ES) 200100266

(51) Int. Cl. A01N 63/00; C12N 1/20

(52) U.S. Cl. 424/93.4; 435/252.1

(57) ABSTRACT

The patent describes a method of selective application of entomopathogenic fungi, characterized by employing an attractant-contaminant device in which the spores of said fungus are fixed on an adsorbent material; this same adsorbent material or another, depending on the case, incorporates a specific attractant and is located on an adherent material. This adherent material can, in certain cases, incorporate a gelling agent and different additives, which maintain the adequate level of humidity for the survival of the spores. Standing out among the advantages of this ecological method of application of entomopathogenic fungi is the selectivity resulting from the use of specific attractants and the long duration of the attractant-contaminant effect thanks to the use of the controlled rate emitter (adsorbent substance) and to the fact that a greater persistence of the spores is achieved with control of the humidity.

### RELATED APPLICATIONS

The present application is a Continuation of co-pending PCT Application No. PCT/ES02/00043, filed Jan. 30, 2002 which in turn, claims priority from Spanish Application Serial No. 200100266, filed on Jan. 30, 2001. Applicants claim the benefits of 35 U.S.C. §120 as to the PCT application and priority under 35 U.S.C. §119 as to said Spanish application, and the entire disclosures of both applications are incorporated herein in their entireties.

### TECHNICAL FIELD OF THE INVENTION

The present invention is set within the agricultural sector, particularly in the field of process for combating insects. More specifically, it refers to a carrier vehicle of spores of at least one entomopathogenic microorganism adsorbed on a granular porous support or in powder form; to a device which includes said composition or said vehicle; to an incorporated attractant semiochemical component and to a method for combating insects by means of the use of said device.

### BACKGROUND OF THE INVENTION

The use of insecticides in controlling plagues of insects presents problems such as toxicity; the lack of selectivity which leads to the destruction of beneficial insects or of natural predators of the plague it is sought to combat; and the resistance developed by the insects which causes an increase in the dose needed to maintain its efficacy.

The use of semiochemical substances has developed substantially and, in particular, techniques such as surveillance in order to anticipate the appearance of plagues and extinguish them, sexual confusion in order to prevent reproduction of the insects, and mass capture in order to decrease the population of them.

Semiochemicals display low toxicity and very high selectivity, since they act on a single species of insect. Also, they do not provoke any resistance among insects or contaminant effects for the environment.

As well as the use of semiochemicals in a process for combating insects, the entomopathogenic properties of certain types of microorganisms, such as fungi, can also be exploited. These properties are known, but their effectiveness in biological control of insect plagues depends largely on their method of application.

For the specific and directed application of entomopathogenic microorganisms, supports are needed for the spores that will keep them viable and without germinating for long periods of time in the field and, moreover, combining them with specific semiochemicals that will attract the particular species to be combated, towards the contaminant support.

For the use of semiochemicals to be effective, it is necessary to have physical supports complying with requisites such as:

providing an adequate emission rate of the semiochemical,

permitting prolonged duration of the emission,

avoiding degradation of the semiochemical,

not producing contaminant waste, and

to be economical and to permit easy application of the semiochemical.

Of the emitter supports existing on the market, such as, for example, rubber septa, polyethylene pipes, porous plastic sheets, etc., none of them complies with the stated requisites.

Moreover, the methods so far used for the application of entomopathogenic microorganisms present problems of survival which diminish their efficacy.

Application WO-9101736 claims a gel of natural polymers to which attractant entomopathogens, feeding stimulants and protectors against UV are incorporated. It also claims several forms of gelling the polymers by means of metal cations and the insects ingest the entomopathogen with the gel.

The differences with the present process are that what is claimed is the adsorption of entomopathogenic fungi spores in adsorbent materials selected among silicates, silicoaluminates, phosphoaluminates and ion exchange resins, which further incorporate the insect-specific volatile attractant compounds (pheromones and other natural or synthetic attractants) in such a way that a slow, controlled and lasting emission of the same takes place (Patent UPV No. P 9701077) and they maintain a suitable degree of humidity for the survival of the spores.

WO-A-9208355 claims a process for drying microbial cultures, mixing them with a carrier like those used for formulating insecticides for dusting and drying, them with air. Its application is by means of coating seeds with microbial dust and is of no use for protecting aerial parts.

Application WO-9211356 claims some particular strains of entomopathogenic fungi. It also claims a formulation wherein the spores are incorporated into an emulsified bait containing cotton seed flour (proteins and carbohydrates), extracts of parts of cotton plants, oil and an emulgent, all these as a “feeding stimulant”. This bait is sprayed in liquid form or it is added to a solid carrier in powder form, in the usual form of insecticide formulations and is applied as a wettable powder or in dusting.

WO-9324013 claims a storage chamber for entomopathogenic fungi for their conservation until they are used for controlling insects such as cockroaches, flies, ants and plagues of larvae, plagues on lawns and caterpillars. In these chambers, the temperature, humidity and oxygen are regulated. An emulgent can also be added for applying the fungus in aqueous suspension or in powder form. It cites two patents for cockroach traps.

WO-9510597 claims a formulation of entomopathogenic fungi wherein the conidia are suspended in a mineral oil. This formulation is used for being applied by spraying on crops. It can be made emulsionable for dispersion in water by the addition of an emulgent or adding an inert carrier to it for its application in the form of a suspendible powder as is usual in insecticide formulations.

EP-A-0406103 claims the culture of an entomopathogenic fungus on an inert solid support such as montmorillonite or atapulgite and the use of the resulting mass for spraying against plagues or for fermenting in the soil under sporulation conditions.

The use of adsorbent materials as controlled rate emitters of insect attractants—generally sexual pheromones—is described WO-A-9944420-A and WO-A-0000446. Nevertheless, the process described in these patent applications does not include the use of an entomopathogenic agent.

As is revealed from the above paragraphs, both the actual existing physical supports and, in general, the methods for combating insects based on the use of entomopathogenic microorganisms still display serious deficiencies in several basic aspects, such as the period of duration of the composition or vehicle used and the demonstrated efficacy, for which reason there exists a demand for the development of new insect control systems.

### DESCRIPTION OF THE INVENTION

The present invention aims to overcome the drawbacks of the state of the art by means of a carrier vehicle for spores of at least one entomopathogenic microorganism, consisting of an adsorbent support selected among a granular form, a powder form and mixtures of them, capable of retaining the spores and maintaining their viability and a attractant semiochemical component, wherein:

the adsorbent support is a material selected among silicates, silicoaluminates, phosphoaluminates, ion exchange resins and combinations of them;

the spores are adsorbed onto the mineral support;

and the semiochemical component, selected among attractant semiochemical substances of insects susceptible of suffering the entomopathogenic effects of the spores, is also adsorbed on that support or on another analogous support.

The advantage of this invention is that it combines three different effects:
a) The fixing of the biological material on a support which adsorbs it on its surface in an adequate way for contaminating insects by contact and for maintaining its viability during a long period of time.
b) This support maintains by adsorption a degree of humidity that is adequate for preventing the biological material from drying out yet is insufficient for its germination, giving a prolonged life.
c) The same or another support, or a mixture of them, adsorbs the attractant producing a controlled and lasting emission thereof and ensuring the specificity.
d) An adhesive fixes the adsorbent support, in powder or granular form, on a solid surface (plates, spheres, etc.). This adhesive can be an organic polymer or an aqueous gel which contributes to maintain a constant and regulated supply of humidity to the adsorbent support located thereon.
e) The local form of application is more specific and ecological than general spraying.
f) The carrier support of spores and/or of attractants is adhered to a solid surface (plates, spheres, etc.) by means of a natural or synthetic polymeric adhesive, and the device is located in the field in the usual way for traps.

A first additional object of the present invention is a composition for combating insects which incorporates that vehicle comprising spores of at least one entomopathogenic microorganism, at least one adsorbent support selected from a granular form or in powder form and an attractant semiochemical component.

A second additional object of the present invention is a device for combating insects, characterized in that it comprises a receptacle comprising, in a way that is accessible for insects, a carrier vehicle of spores of at least one entomopathogenic microorganism, at least one adsorbent support in granular or powder form, and an attractant semiochemical component.

A third additional object of the present invention is a method for combating insects by means of infection of them with spores of at least one entomopathogenic microorganism, characterized in that an efficacious quantity of the vehicle or of the composition obtained, according to the invention, is made available to the insects.

The entomopathogenic microorganism can be any fungus or bacterium capable of contaminating the insects. In a preferred manner, said microorganism is a fungus, for example Metarhizium anisopliae, Paecilomyces fumosoroseus, Beauveria bassiana, etc. The spores of the entomopathogenic microorganism are present in a quantity between 1×10³ and 1×10¹² spores per gram of adsorbent support.

The support is a natural or synthetic adsorbent material selected from among silicates, silicoaluminates, phosphoaluminates, ion exchange resins, or any combination of them. In a preferred manner, said support is a zeolite and more preferably still it is a sepiolite.

The size of the structural channels of the adsorbent support must be adequate for housing the spores yet preventing them from penetrating to the interior of the structure of the support when exposed and accessible to insects. This size of particle lies between 230-450 nm, preferably between 240 nm and 420 nm (FIG. 1). The quantity of adsorbent support used is between 50 and 60 mg per cm³.

The device of the present invention can furthermore include an intermediate base for fixing the adsorbent support on a flat or curved solid surface. This intermediate base can be any organic adherent polymer, such as for example an elastomeric adhesive, or an emulsion of them, or an aqueous adherent gel obtained with one or more natural or synthetic gelling agents, such as for example agar, alginates or other polymers of algae and fungi, carboxymethylcellulose, crystalline cellulose, quitosanes and derivatives, methylcellulose and methylbutylcellulose.

When this adherent intermediate base is an aqueous gel, a wetting agent can also be added to it, such as sorbitol, glycerol, manitol, xylitol and combinations thereof. Desiccation is thereby avoided and this intermediate base also helps to maintain the humidity of the adsorbent support in powder or granular form for the spores.

In a preferred embodiment, methylbutylcellulose is used. In another preferred embodiment, a mixture is used of carboxymethylcellulose (CMC) and methylcellulose (MC) in a proportion of CMC and MC of between 5 and 40% by weight. The moistening agent is selected from among one or more polyalcohols, in a preferred way it is selected from among sorbitol, glycerol, manitol, xylitol and combinations thereof. In a still more preferred way, sorbitol or glycerol is used.

The quantity of said moistening agent in the device is between 20% and 96% by dry weight. It is preferably in a proportion of from 25% to 85% by dry weight of the device.

The attractant semiochemical component is specific for the species being dealt with and can be a pheromone or other natural or synthetic attractant that produces an adequate response. Specific examples of semiochemicals are trimedlure and 1-4-tetramethylenediamine, specific attractants of Ceratitis capitata, or methyl-eugenol, an attractant of Bactrocera dorsalis. The semiochemical component is present in the adsorbent support in a proportion between 0.005-1.0 gram per gram of adsorbent support, preferably in a proportion between 0.02-0.7 grams per gram of said adsorbent support.

The adsorbent support of the present invention can furthermore comprise an oil component selected among mineral oils, vegetable oils, animal oils and mixtures thereof, which contributes to fixing the attractant semiochemical component and protecting the spores. The function of the oil component is to help in the retention of the semiochemical and its slow and controlled emission and to maintain the spores in an oily medium in order to increase their protection and extend their life-time. Said oil component is present in the vehicle by an amount between 20% and 75%.

The device of the present invention can adopt various forms according to the arrangement of its components, thereby ensuring the maximum duration of the attraction and contamination of the insect. So, the surface coated with the support can be flat (plates of different dimensions, folded or unfolded) or curved (spheres imitating fruits, cones, cylinders and other shapes).

In a first form, an adsorbent support containing the spores and the semiochemical component with or without oil is adhered to a solid surface by means of organic adhesives or aqueous gels.

In a second form, the spores and the semiochemical component are adsorbed onto different adsorbent supports. The mixture of the two supports is adhered to a solid surface by means of organic adhesives or aqueous gels.

In a third form, the adsorbent support containing the semiochemical component is incorporated into the adhesive base layer and the adsorbent support containing the spores is spread on the surface.

In a fourth form, the adsorbent support containing the semiochemical component is in the form of a pill, located in the centre of the adhesive base layer and surrounded by the adsorbent support containing the spores.

The vehicle of the present invention can include a UV ray photoprotector in its composition.

For its transportation and use, the vehicle of the present invention is located on an object, for example, plates or spheres, the surface of which contains an adherent component and the adsorbent. These plates are located in the field underneath the traps, which protect them from the sun and rain.

An additional object of the present invention is a composition that includes a carrier vehicle for spores of at least one entomopathogenic microorganism adsorbed on a support, as specified earlier, the application of which can be done in a suspension of the vehicle in a fluid, such as water for example, or by dusting, in an manner analogous to insecticide formulations.

A second additional object of the present invention is a device for combating insects, comprising a receptacle which, in a manner accessible for the insects, comprises a vehicle or a composition which incorporates spores of at least one entomopathogenic microorganism adsorbed on an adsorbent support, as has been specified earlier.

By means of the present invention, a long period of activity of the spores is achieved since the device maintains the necessary humidity in the adsorbent material for their survival and permits the degree of humidity to be maintained for prolonged periods, of the order of 2 to 5 months of exposure in the field.

Another advantage over the prior art is the selective effect that is achieved, thanks to the use of an attractant semiochemical, specific to the species to be dealt with.

Another advantage of the present invention consists of the use of particles of adsorbent material as support for the spores, which grants efficacy to the method of combating insects since it ensures its contact with the insect and contamination of it, as well as acting as an attractant vehicle and so that the humidity can reach the spores.

### EXAMPLES

The following examples serve to illustrate the different aspects of the invention.

### Example 1

An adherent layer of 2 mm of polyisobutylene is deposited on a plastic plate, and on this another layer of MCM-41 zeolite carrier of adsorbed spores of Metarhizium anisopliae suspended in mineral oil is deposited. The zeolitic support is also impregnated with specific attractants of Ceratitis capitata, for example, with trimedlure or 2-4-tetramethylenediamine in a proportion of 0.5 and 0.005 g per gram of zeolite.

Table 1 shows the result obtained with the spore carrier vehicle, exposed in the field for three months. In particular, the effect of ageing is shown on the physical and biological properties of the attractant-contaminant plates of Ceratitis capitata, along with the results of its action on the insects.

TABLE 1

| | Power of Attraction | Mortality | Loss of |
|---|---|---|---|
| Time | (%) ^{a)} | (%) ^{b)} | humidity |
| (days) | (mean ± SD) | (mean ± SD) | (%) |
| 0 | 2.10 ± 1.5 | 15.0 ± 0.9 | 0 |
| 15 | 8.2 ± 0.5 | 45.8 ± 1.6 | 35.4 ± 1.1 |
| 30 | 30.2 ± 2.6 | 68.4 ± 2.7 | 45.1 ± 2.7 |
| 45 | 37.4 ± 2.1 | 70.3 ± 2.7 | 59.7 ± 3.6 |
| 60 | 46.7 ± 1.7 | 76.4 ± 3.4 | 65.4 ± 2.6 |
| 75 | 40.9 ± 0.9 | 70.8 ± 3.2 | 69.3 ± 1.8 |
| 90 | 27.5 ± 2.5 | 67.6 ± 1.8 | 72.3 ± 3.1 |

^{a)} Mean count of males alighting on the plate, every 5 minutes, for 3 hours.
^{b)} Number of dead flies as a result of mycosis following a 24 hour exposure period to the plate.

### Example 2

A plate is prepared with a gelled adherent base of methybutylcellulose in a proportion that can vary from 10 to 45%. Glycerol is added as a moistening agent in a proprtion of 20 to 75% by dry weight. Deposited on the is a layer of sepiolite of particle size between 240-420 nm, which is impregnated with spores of Paecilomyces fumosoroseus, suspended in mineral oil. The attractant semiochemical used is methyl-eugenol, an attractant of Bactrocera dorsalis, which is adsorbed on another portion of sepiolite in a proportion between of 0.8 gram of sepolite and is compacted to form a pill, which is partially submerged in the centre of the plate carrying the gel component.

The plates are located in the field underneath the traps, which protect them from the sun and rain.

Table 2 shows the effect of ageing in the physical and biological properties of the attractant-contaminant plates of Bactrocera dorsalis, along with the results of its action on the insects.

TABLE 2

| | Power of Attraction | Mortality | Loss of |
|---|---|---|---|
| Time | (%) ^{a)} | (%) ^{b)} | humidity |
| (days) | (mean ± SD) | (mean ± SD) | (%) |
| 0 | 6.10 ± 5 | 30.0 ± 0.7 | 0 |
| 15 | 11.2 ± 1.3 | 40.3 ± 1.1 | 30.1 ± 1.6 |
| 30 | 35.4 ± 1.9 | 78.0 ± 2.8 | 45.1 ± 2.7 |
| 45 | 49.8 ± 3.4 | 84.7 ± 3.4 | 43.4 ± 2.4 |
| 60 | 55.9 ± 2.4 | 90.3 ± 2.1 | 46.3 ± 2.6 |
| 75 | 50.2 ± 1.3 | 85.7 ± 1.4 | 50.1 ± 2.2 |
| 90 | 38.3 ± 2.2 | 70.4 ± 0.9 | 62.3 ± 1.3 |

^{a)} Mean count of males alighting on the plate, every 5 minutes, for 3 hours.
^{b)} Number of dead flies as a result of mycosis following a 24 hour exposure period to the plate.

1. A carrier vehicle of spores of at least one entomopathogenic microorganism on an adsorbent support selected among a granular form, a powder form and mixtures thereof, and of an attractant semiochemical component, characterized in that:
the natural or synthetic adsorbent support is a material selected from among silicates, silicoaluminates, phosphoaluminates, ion exchange resins and combinations of them;
the spores are adsorbed onto the mineral support and located on the surface in a way that is accessible to the attracted insects;
and the semiochemical component is selected from among semiochemical substances specifically attractant of the species of insect it is wished to combat and is adsorbed on an adsorbent support.

2. A vehicle according to claim 1, characterized in that it furthermore comprises an adherent intermediate base for retaining the adsorbent powder and preventing its dispersion. This base can be a viscous polymer such as an elastomer insoluble in water or an aqueous gel with a moistening agent. This adsorbent intermediate base is applied on plates, spheres or other solid shapes for being located in the field.

3. A carrier vehicle of spores according to claim 1, characterized in that the spores are suspended in an oil component selected among mineral oils, vegetable oils, animal oils and mixtures of them, and adsorbed on the adsorbent support.

4. A vehicle according to claim 2, characterized in that the attractant semiochemical component is adsorbed on the adsorbent support and said support, coated with the spores, is spread on the intermediate adherent.

5. A vehicle according to claim 2, characterized in that:
the semiochemical component is adsorbed on a natural or synthetic support selected among a granular form, a powder form and mixtures thereof, and selected from among silicates, silicoaluminates, phosphoaluminates, ion exchange resins and combinations of them, and this support can be the same as or different from the spore carrier.

6. A vehicle according to claim 3, characterized in that the spores are suspended in an oil component selected among mineral oils, vegetable oils, animal oils and mixtures of them.

7. A vehicle according to claim 5, characterized in that:
the semiochemical component is adsorbed on the adsorbent support, the same as or different from the spore carrier.
the adsorbent support together with the semiochemical is compacted in pills, which are surrounded by the carrier support for spores. The two adsorbent supports can be the same or different.

8. A vehicle according to claim 3, characterized in that: the spores are suspended in an oil component selected among mineral oils, vegetable oils, animal oils and mixtures of them.

9. A vehicle according to claim 2, characterized in that: the intermediate adhesive layer is selected among organic polymeric adhesives or is an aqueous gel of natural or synthetic polymers.

10. A vehicle according to claim 9, characterized in that: the adherent layer is formed from organic adhesives or from gels, the gelling substance of which is selected among natural or synthetic hydrophilic polymers such as carboxymethylcellulose, crystalline cellulose, carboxymethylchitosane, methylcellulose, methylbutylcellulose, quitosanes, polymers of algae and plants and combinations thereof.

11. A vehicle according to claim 2, characterized in that the moistening agent consists of one or more polyalcohols.

12. A vehicle according to claim 11, characterized in that the moistening agent is selected among sorbitol, glycerol, manitol, xylitol and combinations thereof.

13. A vehicle according to claim 2, characterized in that the moistening agent is present in a quantity between 20% and 95% of the dry weight of the gelling agent.

14. A vehicle according to claim 1, characterized in that the semiochemical component comprises at least one pheromone or a synthetic or natural attractant, specific for the species is it wished to combat.

15. A vehicle according to claim 1, characterized in that the semiochemical component is present in the adsorbent support in a quantity between 0.005 and 1 g per gram.

16. A vehicle according to claim 5, characterized in that the semiochemical component is present in the second adsorbent support in a quantity between 0.0005 and 1 g per gram of said second adsorbent support.

17. A vehicle according to claim 1, characterized in that the entomopathogenic microorganism can be any fungus or bacterium capable of contaminating the insects.

18. A vehicle according to claim 1, characterized in that the spores of the entomopathogenic microorganism are present in a quantity between 1×10³-1×10¹² spores per gram of adsorbent support.

19. A vehicle according to claim 3, characterized in that the oil component is present in a quantity between 30-75% per gram of adsorbent support.

20. A composition for combating insects which contains a vehicle as defined according to claim 1.

21. A device for combating insects, characterized in that it comprises a receptacle containing, in a form accessible for the insects, a vehicle defined according to claim 1.

22. A device for combating insects, characterized in that it comprises a receptacle containing, in a form accessible for the insects, a composition defined in claim 20.

23. A method for combating insects by means of infection of the insects with spores of microorganisms, characterized in that an efficacious quantity of spores is made available to the insects, according to any of claims 1.
